(19) United States
(12) Patent Application Publication
(10) Pub. No.: US 2010/0088793 A1
(43) Pub. Date: Apr. 15, 2010

(54) Face and antiglare protection device for persons working in sites involving hazardous, damaging or disturbing radiation, especially welders

(76) Inventors: Livio Ghisleni, Zurich (CH); René Werthmüller, Ammerzwil (CH)

Correspondence Address: THE NATH LAW GROUP, 112 South West Street, Alexandria, VA, 22314, US

(21) Appl. No.: 12/308,087

(22) PCT Filed: Jun. 6, 2007
(86) PCT No.: PCT/CH2007/000282

§ 371 Date: Nov. 6, 2009

(30) Foreign Application Priority Data
Jun. 6, 2006 (CH) 0902/06

(51) Int. Cl. A61F 9/06

(52) U.S. Cl. 2/8.2; 348/135; 348/53; 345/419; 382/162; 704/251

(57) ABSTRACT

The invention relates to a face and antiglare protection device (1) for observing an object field or a workplace with light intensity differences varying in time and/or space of, for example, 10000:1, as in welding operations. The device (1) comprises at least one camera (4) for generating electronic image signals, at least one sensor (5) for generating electronic measuring data, at least one electronic system (8) for processing the image signals and the measuring data and at least one display for the optical representation of the object field and the measured data. According to the invention, the device comprises at least two electronic cameras (4) having a different spectral and/or spatial resolution. The electronic system (8) is provided with image analyzing means which analyze the image signals captured by said electronical cameras (4) and, preferably, the measuring data of the at least one sensor (5). The electronic system (8) further comprises means of generating a virtual image of the object field, wherein said means synthesize the analyzed image signals to form a virtual image such that the entire object field can be recognized in detail on the display by the human eye irrespective of its spectral and topological conditions.

The present invention concerns a face and antiglare protection device according to the preamble of claim 1.

Protection devices for the observation of a workplace where dangerous splashes and/or sparks as well as powerful light intensity variations occur—such as are typical during welding—are well known. These devices generally have a non-transparent facial protection in the form of a mask, a helmet or a shield and have a viewing window which is provided with a suitable glare shield—typically a passive or an active optical filter. Passive optical filters include darkened, and in particular doped, glass or plastic panes which block and/or polarize infrared or ultraviolet radiation, whilst active optical filters essentially comprise so-called ADFs (Automatic Darkening Filters) for industrial welders. These automatically darkening filters have electro-optical components, and in particular at least one liquid crystal cell and a light-sensitive sensor by means of which the transmission value of the optical filters is automatically adjusted to the desired value. The suitable electronics for adjusting the transmission values of an optical filter is described, for example, in European Patent 1 190 689. The configuration of such an optical filter is known, for example, from U.S. Pat. No. 5,930,047.

Further embodiments of these face and antiglare protection devices comprise additional sensors or measuring instruments, whose signals are transmitted to an electronic circuit in the face and antiglare protection device and whose measurement readings are visible in the field of vision of the user. The signals are transmitted by means of cables or by means of a sender/receiver system. The electronics in the face and antiglare protection device evaluates the signals received and controls a display device so as to make the detected measurement values visible to the user. These display devices can be simple, possibly color-coded light diode arrays or can be LCD-displays. Relevant values for the user can be the temperature within the immediate work area, the actual intensity of current of the welding tool or the welding time. Exemplary further embodiments are described in European Patent 0 963 744 or WO-01/12376.

In U.S. Pat. No. 6,230,327 it has been suggested to provide a face and antiglare protection device with a camera whose image is displayed on an LCD-monitor (display) arranged above the viewing window. By choosing a suitable range for the wavelength sensitivity of the camera, for example a close infrared-range of between 800 to 1100 nm, more details can be made perceptible, for example in the region of the weld pool. These regions are not visible with conventional equipment because they filter out the harmful infrared and ultraviolet regions.

Unfortunately these types of face and antiglare protection devices are not very user-friendly, because although the different displays, image sections and/or detailed views are within the viewing field of the user, they are displayed in varying viewing directions. Furthermore, the suggested solutions have been shown to be particularly heavy, susceptible to breakdown, that is, they are undependable and/or expensive and thus they are not suitable for the market.

Publication WO-2005/102230 describes a welder's helmet equipped with a number of cameras instead of a viewing window. These cameras allow the work field to be observed and recorded from varying viewing directions. The user of such a helmet is free to choose the display of any one of these cameras and can therefore observe regions which would otherwise not be viewable by just turning his head. This welder's helmet uses the display and the electronic cameras in a conventional manner, i.e. it cannot compensate for the extreme variations in light intensity which occur during welding. The images produced with this welder's helmet do not allow for a detailed field of vision on to the actual work place.

It is therefore the aim of the present invention to provide a face and antiglare protection device which does not have the disadvantages of the known devices. In particular, it is the aim of the present invention to provide an economic and light weight face and antiglare device which can display in detail an object field having extreme light intensity variations of, for example, 10,000:1 or more in one and the same field of viewing direction.

According to the invention, this problem is solved by a face and antiglare device with the features of claim 1, and in particular by a face and antiglare device suitable for a welder for observing an object field or work place having temporally and spatially varying light intensity variations, said device having at least one electronic camera for generating electronic image signals, at least one sensor for generating electronic measuring data, at least one electronic system for processing the image signals and measuring data, as well as at least one display for the optical representation of the object field and the measured data, wherein said device comprises at least two electronic cameras having differing spectral and/or spatial resolution, whereby the electronic system comprises means for image analysis, said image analysis means analyzing the image signals captured by said differing electronic cameras and preferably analyzing the measuring data of the at least one sensor, whereby the electronic system comprises means for generating a virtual image of the object field, said image generating means synthesizing the analyzed image signals to create a virtual image in such a manner that the entire object field can be recognized in detail on the display by the human eye, irrespective of its spectral and topological conditions.

The varying light sensitivity of the image sensors of the individual electronic cameras is predetermined by using different semiconductor materials, in particular silicium (silicon), gallium or germanium. The cameras of the face and antiglare device according to the invention preferably have miniature optics. The electronics for processing the image signals and measuring data comprises means for generating a virtual image of the object field.

In a preferred embodiment the face and antiglare device according to the invention has a display for each eye and the electronics comprises means for generating a three-dimensional virtual image (Virtual Reality Image) of the object field so as to portray the topological situation.

Further embodiments of the face and antiglare device according to the invention have electronics with means for speech recognition and/or with means for generating acoustic signals. It is understood that the electronics can also control an additional light source, in particular for illuminating portions of the object field in a particular temperature and/or luminosity range. The electronics can also be provided with means for storing user-specific settings such as interocular (interpupillary) distance, acuity of vision, coloring of the display and so forth, or with means for controlling wireless transmission of data signals.

The advantages of the present invention are immediately evident to the expert and are to be seen in particular in the use of miniaturized VR-image technology in the field of antiglare technics. By means of this image technology it is possible to synthesize (a number of) images into one virtual image in such a manner that regions of the object field having unusually high light intensity (arc and/or weld pool) and regions of the object field having unusually low light intensity (nocturnal environment) can be displayed in detail in the same image, thus providing the user with maximum information about his work place. The same purpose is served by the display of the object field, which allows the topological conditions of the work place to be depicted three-dimensionally. This improves not only the quality and quantity of work done but also increases the operational safety for the user. Miniaturization leads to a considerable decrease in weight and increase in ease of use. Furthermore, the face and antiglare protection device according to the invention permits the use of low-cost standard components.

The invention shall be more closely described with the aid of an example and the drawing.

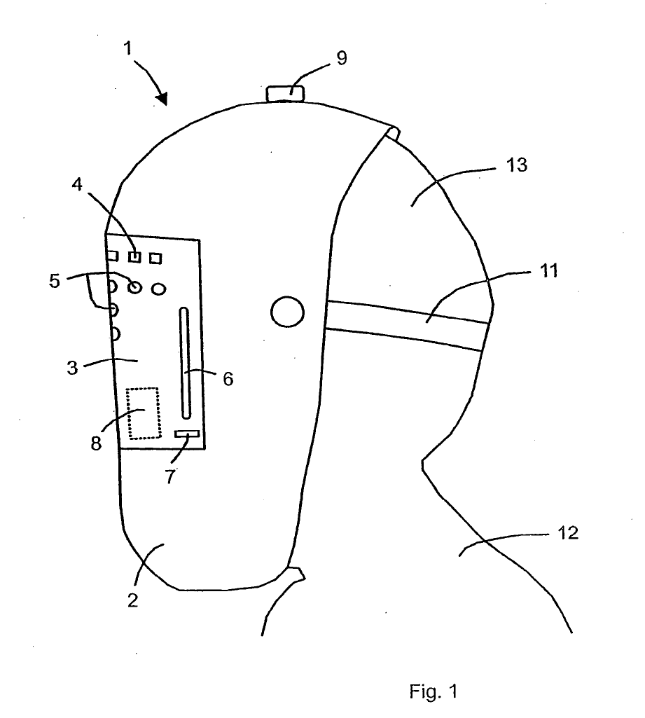

FIG. 1 shows a schematic illustration of a face and antiglare protection device according to the invention.

FIG. 1 is a schematic illustration of a face and antiglare protection device 1 according to the invention. This essentially comprises an area for the face shield 2 and an area for the glare shield 3. The face and antiglare protection device 1 is preferably made of a heat and scratch resistant material and, in the embodiment shown, may be secured to the head 13 of a user 12 by means of a fastening strap 11. It is understood that this face and antiglare protection device 1 may be designed in the form of a mask, a helmet or some other protective covering. In the embodiment shown, the glare shield 3 of the face and antiglare protection device 1 is integrated into the face shield 2 of the face and antiglare protection device 1, but it can also be detachably connected to the face shield 2. In the following, an integrated zone in the eye region of the face shield 2 shall therefore also be termed glare shield 3. According to the invention the glare shield 3 does not permit a direct view of the object to be observed. This ensures that no damaging radiation reaches the eyes of the user 12. In order to give the user 12 a perfect view of the field of work or of the object field, the glare shield 3 is provided with at least two different cameras 4 which view the field of work with different spectral and/or spatial resolutions. These two cameras 4 comprise miniaturized image sensors and miniature optics such as are well known, for example, from today's widely used mobile telephones with camera function. According to the invention, both these image sensors are connected to electronics 8 which analyses these images and compiles a virtual picture which is recognizable in detail to the human eye. In a preferred embodiment, an individual display is provided for each eye, thereby creating a three-dimensional image of the object field for the user. The image signals of the image sensors of these cameras 4 are conducted to electronics 8 which processes these signals in an appropriate manner and synthesizes them to a three dimensional picture. Such electronic systems are well known and are described, for example, in U.S. Pat. No. 6,084,557 or WO-2004/084118. With these systems an object field is captured by means of electronic cameras and the image signals are compared to virtual images and formatted to a new VR-image (Virtual Reality Image) before being made available to the user. According to the invention, the image signals of the cameras 4 are processed in such a way that the user 12 can view the object field—which may have variations in brightness or in light intensity of 10,000:1 or more—in an overall picture having a resolution suitable for the human eye, i.e. the electronics 8 recognizes the extreme differences in light intensity and reduces these to a level suitable for the human eye. Further image sensors 5 having varying degrees of sensitivity are provided to support the electronics 8. Generating a VR-image is within the normal technical scope of the expert and is not the object of the present invention.

It is understood that the electronics 8 can be provided with additional software for image analysis which may permit individual object elements to be colored or flagged during or after the work step, for example in order to indicate temperature values in certain areas of the object field to the user.

In order to better determine the topological conditions of the work area it is advantageous to use camera settings different from those for imaging the arc, in particular different focal lengths, apertures, focuses or sensor sensitivities. These settings can be adjusted by motor-driven or manual adjustment means directly on the camera. The camera sensors are usually silicium-(silicon-)based. In conjunction with other basis materials such as, for example, gallium or germanium, it is possible to determine other spectrums, i.e. other temperature ranges. With a combination of sensors having differing basis materials in two or more camera sensors it is possible to obtain an exact temperature analysis of the object field and to display these electronically.

In a further embodiment of the present invention, the electronics 8 enables important information (relevant parameters for work control or time functions such as, for example, amperage, welding time etc.) to be displayed which have previously been determined by the cameras 4 and/or by the sensors 5. This additional information can be accessed either as text and/or as symbols and can be edited with the aid of an external processor or electronic memory. This external processor or memory communicates with the electronics 8 either by glass fiber or wireless. A receiver 6 and a transmitter 7 are provided in the face shield 2 for wireless transmission of data or image signals. The processor and/or the memory can either be carried by the user or can be installed on a protected trolley to trail the user. In essence, individual parts of the device, and in particular the power supply, can be attached to a carry-strap instead of being attached to the face and glare shields 2, 3.

The image quality can be improved by the use of an additional light source (any type of lamps, LEDs or laser) at the work place and/or work environment. This light source 9 is preferably attached to the face shield 2.

It is understood that the present face and antiglare protection device can also be provided with a microphone, with speech recognition software, with a loudspeaker and so on, in order to be able to use spoken commands such as “start” or “stop” for controlling the device. Acoustic signals can also be used for device control.

Further developments to the present invention are within the scope of the expert, in particular concerning devices for geometrically adjusting the individual oculars (interocular distance, personal acuity of vision and so forth). These individual geometrical adjustments can be made to the camera sensors, to the readout-displays or to other parts with the aid of adjustable screws, goosenecks etc. The face and antiglare protection device according to the invention can be further personalized by means of automated user adjustments or by mechanical identification means.

It is understood that the use of the present invention is not limited to welding technology but can also be used in other areas of work, such as, for example, fire-fighting or police work, in fact everywhere where poor visibility can hamper the task at hand. In particular, the present face and antiglare protection device can be provided with a miniaturized night vision device.

1. Face and antiglare protection device (1) for observing an object field or a workplace with light intensity differences varying in time and/or space, in particular for welders, said device (1) having at least one electronic camera (4) for generating electronic image signals, at least one sensor (5) for generating electronic measuring data, at least one electronic system (8) for processing the image signals and measuring data, as well as at least one display for the optical representation of the object field and the measured data,
wherein said device comprises at least two electronic cameras (4) having differing spectral and/or spatial resolution, whereby the electronic system (8) comprises means for image analysis, said image analysis means analyzing the image signals captured by said differing cameras (4) and preferably analyzing the measuring data of the at least one sensor (5), whereby the electronic system (8) comprises means for generating a virtual image of the object field, said image generating means synthesizing the analyzed image signals to create a virtual image in such a manner that the entire object field can be recognized in detail on the display by the human eye, irrespective of its spectral and topological conditions.

2. Face and antiglare protection device (1) according to claim 1, wherein said device (1) comprises an individual display for each eye.

3. Face and antiglare protection device (1) according to claim 2, wherein said electronic system (8) has means for generating a three-dimensional virtual image (Virtual Reality Image) of the object field.

4. Face and antiglare protection device (1) according to claim 1, wherein the image sensors are made of different semiconductor materials, in particular silicium, gallium, germanium or combinations thereof.

5. Face and antiglare protection device (1) according to claim 1, wherein said cameras (4) have miniature optics.

6. Face and antiglare protection device (1) according to claim 3, wherein the image analysis means have electronic means for determining the spatial resolution, and/or for determining luminosity distribution and intensity, and/or for determining color and temperature distribution, and/or for determining spectral distribution.

7. Face and antiglare protection device (1) according to claim 1, wherein the electronics has means for language recognition.

8. Face and antiglare protection device (1) according to claim 1, wherein the electronics has means for generating acoustic signals.

9. Face and antiglare protection device (1) according to claim 1, wherein the electronics has means for controlling at least one additional lighting source (9) and/or another peripheral apparatus such as, for example, a fresh air assembly.

10. Face and antiglare protection device (1) according to claim 1, wherein the electronics has means for storing user-specific settings.

11. Face and antiglare protection device (1) according to claim 1, wherein the electronics has means (6, 7) for wire- and/or fibre-linked and/or wireless transmission of data signals.
